(19) United States
(12) Patent Application Publication
(10) Pub. No.: US 2007/0060639 A1
(43) Pub. Date: Mar. 15, 2007

(54) Compositions and methods for intranasal delivery of tricyclic cannabinoids

(75) Inventor: Daniel P. Wermeling, Lexington, KY (US)

Correspondence Address: HARNESS, DICKEY, & PIERCE, P.L.C, 7700 BONHOMME, STE 400, ST. LOUIS, MO, 63105, US

(73) Assignee: University of Kentucky, Lexington, KY (US)

(21) Appl. No.: 11/515,607

(22) Filed: Sep. 5, 2006

Related U.S. Application Data
(60) Provisional application No. 60/715,940, filed on Sep. 9, 2005.

(51) Int. Cl. A61K 31/353

(52) U.S. Cl. 514/454

(57) ABSTRACT

A pharmaceutical composition for intranasal administration to a human or non-human subject is provided, comprising a therapeutically active component that comprises at least one tricyclic cannabinoid in a liquid to semi-solid medium that comprises a pharmaceutically acceptable solubilizing agent in an amount effective to solubilize the cannabinoid. An amount of the composition intranasally administrable as a single dose, upon intranasal administration in a rat model, provides a systemic plasma cannabinoid concentration (i) that, at least at one time point during a period from about 15 minutes to about 2 hours after said administration, is at least about 0.5 ng/ml, but (ii) that at no time exceeds about 100 ng/ml.

This application claims the benefit of U.S. provisional patent application Ser. No. 60/715,940, filed on Sep. 9, 2005, the entire disclosure of which is incorporated by reference herein.

### FIELD OF THE INVENTION

This invention relates generally to pharmaceutical drug compositions and methods for intranasal delivery of cannabinoids. This invention also relates to pharmaceutical drug delivery devices for the intranasal administration of cannabinoids.

### BACKGROUND OF THE INVENTION

Cannabinoids include naturally occurring active compounds in plants such as Cannabis sativa (hemp or marihuana). Synthetic cannabinoids have also been prepared. Exemplary cannabinoids include tetrahydrocannabinols, for example, delta-9-tetrahydrocannabinol (Δ⁹-THC or dronabinol), delta-9-tetrahydrocannabinol propyl analogue, cannabidiol, cannabidiol propyl analogue, cannabinol, cannabichromene, cannabichromene propyl analogue, nabilone and cannabigerol. A number of cannabinoids, more particularly tricyclic cannabinoids such as Δ⁹-THC, are psychoactive. Use of cannabinoids has included inhalation from smoking marihuana plant parts containing relatively high concentrations of cannabinoids, principally Δ⁹-THC. Absorption of cannabinoids from smoking is fast and efficient; however there are a number of drawbacks, aside from issues of illegality, which prevent this delivery method from being ideal. For example, marihuana tar contains similar carcinogens to tar from tobacco cigarettes, but each marihuana cigarette may be more harmful than a tobacco cigarette since more tar is inhaled and retained when smoking. Hashibe et al. (2002), J. Clin. Pharmacol. 42(11 Supp.):103S-107S.

Historically, cannabinoids have been used to treat pain, glaucoma and nausea, to alleviate various mental disorders, as a sedative, and as an anti-emetic. A substantial amount of research performed in the last several decades has provided some evidence that cannabinoid preparations may be useful in treating a variety of conditions including multiple sclerosis and pain. Dronabinol formulated in sesame oil has been approved by the U.S. Food and Drug Administration (FDA) to treat nausea and vomiting associated with chemotherapy in cancer patients and to treat loss of appetite in AIDS patients. Dronabinol is administered orally as Marinol® capsules of Unimed Pharmaceuticals, Inc. Physicians Desk Reference, 59th Ed. (2005), pp. 3248-3250. Nabilone, a synthetic cannabinol, has been used as an anti-emetic in patients receiving chemotherapy and has been investigated for other potentially therapeutic uses. Martindale: The Extra Pharmacopoeia, 29th Ed. (1989) pp. 1553-1554.

U.S. Patent Application Publication No. 2003/0100602 proposes oral administration of dronabinol to stimulate appetite and reduce weight loss in patients suffering from HIV infection. Routes of administration other than oral, or dosage forms suitable for such routes, are mentioned, including suppositories, intranasal administration, transdermal administration, inhalants, sublingual administration and injection.

While oral administration of cannabinoids can be therapeutically useful, there are still challenges associated with oral use. Drugs when swallowed are absorbed by blood perfusing the gastrointestinal tract. This blood flows through the hepatic portal vein into the liver, and in the liver the drug can be metabolized, a process known as first pass metabolism. When a drug is ingested in an active form, often a substantial portion of it is metabolized to an inactive counterpart. This is true of many orally administered cannabinoids including Δ⁹-THC where only 10% to 20% of an orally administered dose typically reaches the systemic circulation in an active form.

Oral delivery of cannabinoids presents a further challenge: when patients ingest the same dosage of a cannabinoid such as Δ⁹-THC, substantial variation can occur from one patient to another in the extent of absorption. Patients that absorb greater amounts of the drug or have lesser capacity to metabolize the drug in their liver may experience adverse effects such as the psychotropic effects associated with smoking marihuana. Patients that absorb less of the drug or have a greater capacity to metabolize the drug may experience diminished therapeutic benefits or no benefit at all.

Such variation in effects reflects the high variability in plasma levels of the active forms. Depending on the dose, Δ⁹-THC may be detected in the plasma after about 30 to about 90 minutes post ingestion, reach a maximum level after about 2 to about 3 hours, and persist in the plasma for about 4 to about 12 hours. The effects of Δ⁹-THC administered orally may not be experienced for some considerable time after administration; thus, in order to prevent nausea, for example, Δ⁹-THC must be taken chronically.

U.S. Patent Application Publication No. 2003/0021752 of Whittle & Guy attempts to address this problem. This publication discusses a mucosal delivery system for lipophilic compositions of a cannabinoid using an emulsion capable of adhering to a mucosal surface. Lipophilic drugs are reportedly absorbed through the mucosal surface.

The highly hydrophobic nature of many cannabinoids creates a challenge for anyone trying to formulate cannabinoid compositions for any delivery method. Traditional delivery methods used for water soluble drugs work inefficiently for cannabinoids and tend to produce the variability in effects discussed above. For example, Δ⁹-THC when administered orally can exhibit erratic bioavailability.

Intranasal administration has been proposed in efforts to resolve these insufficiencies.

U.S. Pat. No. 4,464,378 to Hussain proposes preparing a nasal dosage form of Δ⁹-THC by suspending the drug in an aqueous system.

U.S. Pat. No. 6,380,175 to Hussain et al. proposes a method for enhancing delivery of Δ⁹-THC by intranasal administration of a water-soluble pro-drug.

U.S. Patent Application Publication No. 2003/0003113 of Lewandowski proposes administration of addictive drugs including cannabinoids as part of a method of cessation therapy. Routes of delivery including transdermal, intranasal and sublingual administration are proposed.

U.S. Patent Application Publication No. 2002/0077322 of Ayoub proposes use of cannabinoids for protection against glutamate-induced injury, and mentions nasal administration as a possible method of delivery.

U.S. Patent Application Publication No. 2004/0186166 of Burstein et al. proposes use of cannabinoids for treatment of disorders involving peroxisome proliferator-activated receptor gamma (PPARγ), and mentions nasal administration as a possible method of delivery.

Stinchcomb et al. (2004), XIV Symposium, International Cannabinoid Research Society, reported evaluation of intranasal delivery of cannabidiol in rats.

International Patent Application Publication No. WO 2005/044093 of Zajicek proposes use of Δ⁹-THC for treatment of multiple sclerosis, and mentions nasal administration as a possible method of delivery.

Pylak et al. (1999), Soc. Neurosci. Abstr. 25(1):924, report that when Δ⁹-THC was administered intranasally at 1.14-1.33 mg/kg in a rat model, an analgesic response was observed during the period from 15 to 120 minutes after administration. Data are presented comparing the analgesic effect with effects of ethanol and anandamide.

There remains a need for pharmaceutically acceptable compositions suitable for intranasal delivery of cannabinoids.

### SUMMARY OF THE INVENTION

There is now provided a pharmaceutical composition comprising a therapeutically active component that comprises at least one tricyclic cannabinoid, in a liquid to semi-solid medium that comprises a pharmaceutically acceptable solubilizing agent in an amount effective to solubilize the cannabinoid. The composition is intranasally administrable to a human or non-human subject. Upon intranasal administration of 10 μl of the composition per nostril in a rat model, a systemic plasma cannabinoid concentration is obtained (i) that, at least at one time point during a period from about 15 minutes to about 2 hours after said administration, is at least about 0.5 ng/ml, but (ii) that at no time exceeds about 100 ng/ml.

There is also provided an apparatus comprising (a) a reservoir containing a sprayable liquid composition having characteristics as described immediately above, (b) an atomization device configured for insertion in a nostril, and (c) means for actuating the device to deliver droplets of the composition to the nostril.

There is further provided a method for delivering a tricyclic cannabinoid to a subject, the method comprising intranasally administering a composition as described above.

There is still further provided a method for treatment or prevention of a cannabinoid receptor mediated condition or disorder, the method comprising intranasally administering to a subject a therapeutically effective amount of a composition as described above.

Further features and benefits of the invention will be apparent to one skilled in the art from reading this specification.

### DETAILED DESCRIPTION

Cannabinoids are chemicals typical of and found in the cannabis plant, though these and related chemicals can also be synthesized. According to the invention, the pharmaceutical composition comprises at least one tricyclic cannabinoid.

A “tricyclic cannabinoid” herein is a cannabinoid compound comprising a dibenzopyran substructure 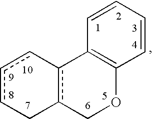
optionally substituted at one or more of the 1, 3, 6 and 9 positions. In one embodiment the composition comprises a compound of formula (I): 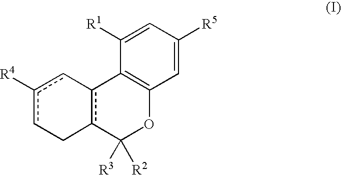
where R¹ is H, OH or C₁₋₃ alkoxy; R² and R³ are independently C₁₋₃ alkyl; R⁴ is C₁₋₃ alkyl, C₁₋₃ alkoxy or a —(CH₂)ₘCOOH or —(CH₂)ₘCHO group where m is an integer of 0 or 1; and R⁵ is a moiety —X—R⁶ where X is CH₂, C(CH₃)₂ or C(O) and R⁶ is C₂₋₈ alkyl, alkenyl or alkynyl or C₃₋₈ cycloalkyl. Illustratively in the compound of formula (I), R¹ is OH, R² and R³ are methyl, R⁴ is methyl or —COOH and R⁵ is a straight or branched chain alkyl, alkenyl or alkynyl moiety having a total of 3 to 10 carbon atoms.

Illustrative tricyclic cannabinoids include cannabinol, tetrahydrocannabinol, Δ⁹-THC, Δ⁸-THC, Δ⁶-THC, Δ¹-THC, THC isomers, Δ⁹-tetrahydrocannabinoic acid, 4″,5″-bisnor-Δ¹-THC-7,3″-dioic acid, levonantradol, nabilone, dexanabinol, ajulemic acid, cannabivarin, tetrahydrocannabivarin, cannabinolic acid, Δ¹-3,4-trans-THC acid, HU210, HU211, derivatives thereof, and prodrugs thereof. Cannabinoid derivatives include 11-hydroxy derivatives, 3-(1′,1′-dimethylheptyl) derivatives, 9-substituted derivatives, 1′-substituted derivatives, propyl analogues, deoxy derivatives, and prodrug ester derivatives, for example of Δ⁹-THC and Δ⁸-THC. Other derivatives include cannabinoid analogues with aliphatic side chains, such as heptynyl, heptenyl, octynyl, octenyl, bromohexynyl, bromohexenyl, nonynyl, and other side chains with double or triple bonds.

According to some embodiments, the at least one tricyclic cannabinoid comprises a hydrophobic tricyclic cannabinoid, for example a highly hydrophobic tricyclic cannabinoid such as Δ⁹-THC. In various embodiments the tricyclic cannabinoid has an octanol-water partition coefficient of at least about 1000:1, at least about 2000:1 or at least about 5000:1 at pH 7. Some tricyclic cannabinoids can be extracted from the cannabis plant. The compounds can also be prepared synthetically. For example, the composition can comprise a synthetic tetrahydrocannabinol, e.g., synthetic Δ⁹-THC. A drug extracted from plants is likely to contain impurities and its potency may vary; when prepared synthetically, a drug typically is more uniform in potency and accordingly more reliable. When considered in view of a narrow therapeutic window, as is the case with many cannabinoids including Δ⁹-THC, good control of potency is very important. This control is often best achieved by use of a synthetic form of the cannabinoid as opposed to a botanical extract.

There are at least two types of cannabinoid receptors: CB1 receptors which are expressed in CNS tissue, and CB2 receptors which are mainly expressed peripherally. Some cannabinoids, including Δ⁹-THC, are relatively non-selective and bind to both receptors. Other cannabinoids have selective behavior in terms of their preference for either CB1 or CB2. In some embodiments, the at least one cannabinoid is a CB1 receptor selective agonist. In other embodiments, the at least one cannabinoid receptor is a CB2 receptor selective agonist. CB2 receptors are G-protein-coupled cannabinoid receptors, and are implicated in immune function.

Typically only one tricyclic cannabinoid is present in the composition in a therapeutically effective amount. Optionally, two or more cannabinoids, at least one of which is tricyclic, are present in a therapeutically effective total amount. Optionally, the therapeutically active component further comprises a second drug that is a non-tricyclic cannabinoid, e.g., cannabidiol, or is not a cannabinoid. The second drug may co-act with the tricyclic cannabinoid in providing the therapeutic benefits described herein. For example, the therapeutically active component may comprise a tricyclic cannabinoid in combination with dexamethasone to provide a composition that may be used as an anti-emetic.

A composition adapted for intranasal administration according to the invention is of particular interest for a tricyclic cannabinoid that is psychotropic above a threshold systemic plasma concentration. Δ⁹-THC is an example of such a cannabinoid. It is believed that intranasal administration, by avoiding a high initial spike in plasma cannabinoid concentration, as occurs for example when the drug is administered intravenously or to a lesser extent when the drug is absorbed by smoking marihuana, while at the same time avoiding first-pass metabolism, as occurs when the drug is administered orally, can minimize psychotropic side-effects while maintaining a therapeutically effective plasma concentration for several hours.

Δ⁹-THC is a highly hydrophobic compound with an octanol-water partition coefficient of about 6000:1 at pH 7. In view of such hydrophobicity and the fact in that the present composition the cannabinoid is in solubilized form, a solubilizing agent is a critical component. The solubilizing agent can comprise a solvent system for the cannabinoid, and this solvent system, itself comprising one or more solvents, can form the bulk of the medium in which the cannabinoid is dissolved. Alternatively, the medium in which the cannabinoid is solubilized can be predominately aqueous and the solubilizing agent can comprise an amphiphilic compound that helps maintain the compound in solubilized form in such a medium, for example as a colloidal solution, emulsion or microemulsion. Optionally, the solubilizing agent can comprise more than one compound, for example at least one solvent and at least one amphiphilic agent. In one embodiment, the composition comprises a simple solution of the cannabinoid in a solvent system such as propylene glycol, alone or in combination with ethanol. Where the cannabinoid is water-soluble, water can be a suitable solubilizing agent. In another embodiment, the composition is in the form of an emulsion or microemulsion wherein the cannabinoid is in solution in a solvent system, for example sesame oil and/or other plant oils, which in turn is emulsified in an aqueous medium in the presence of one or more amphiphilic agents.

Regardless of the nature of the solubilizing agent and whether it comprises one or more compounds, a sufficient quantity of the solubilizing agent is present to solubilize essentially all of the cannabinoid.

The solubilizing agent must be pharmaceutically acceptable when present in an amount needed to solubilize the cannabinoid. For example, the solubilizing agent should not be toxic to nor cause excessive irritation of tissues lining the nasal cavity. For this reason, certain powerful solvents should not be used except as a minor component of the solubilizing agent. Ethanol in particular, when used at high concentrations to deliver a drug to a mucosal surface, provokes a stinging sensation and is beyond the limit of tolerability. See above-cited U.S. Patent Application Publication No. 2003/0021752.

Suitable solubilizing agents for a hydrophobic tricyclic cannabinoid such as Δ⁹-THC include pharmaceutically acceptable glycols. Examples of glycols include but are not limited to propylene glycol, 1,3-butanediol, polyethylene glycol, propylene glycol fatty acid esters, diethylene glycol monoethyl ether and mixtures thereof. Optionally, the solubilizing agent can further comprise ethanol. For example, propylene glycol and ethanol can be present in a volume ratio of at least about 80:20, for example at least about 90:10 or at least about 95:5. According to other embodiments, the solubilizing agent is essentially free of ethanol.

When a hydrophobic tricyclic cannabinoid is present in an aqueous medium, the solubilizing agent can comprise at least one amphiphilic compound in an amount effective to solubilize the cannabinoid in the aqueous medium. For example, the at least one amphiphilic compound can be a cationic, anionic or nonionic surfactant. Illustrative amphiphilic compounds are benzalkonium chloride, benzethonium chloride, cetylpyridinium chloride, dioctyl sodium sulfosuccinate, nonoxynol 9, nonoxynol 10, octoxynol 9, poloxamers, polyoxyethylene (8) caprylic/capric mono- and diglycerides, polyoxyethylene (35) castor oil, polyoxyethylene (20) cetostearyl ether, polyoxyethylene (40) hydrogenated castor oil, polyoxyethylene (10) oleyl ether, polyoxyethylene (40) stearate, polysorbate 20, polysorbate 40, polysorbate 60, polysorbate 80, propylene glycol laurate, sodium lauryl sulfate, sorbitan monolaurate, sorbitan monooleate, sorbitan monopalmitate, sorbitan monostearate and tyloxapol, or mixtures thereof.

Certain cannabinoids including tetrahydrocannabinols such as Δ⁹-THC are susceptible to oxidative degradation (though more so to photodegradation—see, for example, Fairbairn et al. (1976) J. Pharm. Pharmac. 28:1-7), and it is generally preferred to provide a solubilizing agent and/or other ingredients of the composition that minimize exposure of the cannabinoid to oxygen, peroxides or other oxidatively reactive substances. Alternatively or in addition, one or more antioxidants can be included in the composition. Thus in one embodiment ingredients for the composition, in particular the solubilizing agent, are selected providing a sufficiently low level of such substances and/or a sufficiently high level of one or more antioxidants to result in zero to an acceptably low degree of oxidative degradation of the cannabinoid under normal storage conditions in a sealed lightproof container. What constitutes an acceptably low degree of oxidative degradation will depend on particular commercial considerations, but illustratively oxidative degradation of the cannabinoid is not greater than about 5%, for example not greater than about 2% or not greater than about 1%, over a storage period of about 30 days, for example about 90 days, about 180 days, about 1 year or about 2 years.

The composition is adapted for intranasal administration. This means that the composition is in a form physically suitable for intranasal delivery of a therapeutic agent. In one embodiment, the composition is in the form of a sprayable liquid. In other embodiments, the composition is in a semi-solid form, for example, a cream, a gel or an ointment. Without being held to a particular theory, it is believed that most of the absorption of the cannabinoid when administered intranasally is through the nasal mucosa.

According to some embodiments, the tricyclic cannabinoid, e.g., Δ⁹-THC, is present in the composition at a concentration of at least about 1 mg/ml. For example, the cannabinoid can be present in the composition at a concentration of about 1 to about 200 mg/ml, about 2 to about 100 mg/ml, or about 5 to about 50 mg/ml.

As used herein, the phrase “an amount of the composition intranasally administrable as a single dose” means a total volume of the composition that can suitably be administered to one or both nostrils of a human or non-human subject to provide a single dose of the cannabinoid. Such an amount is a practical volume; not so small as to be incapable of administration by any known device, but not so great that a substantial portion of the dose is not retained in the nostrils. For example, with respect to a sprayable formulation intended for administration to a human subject in two aliquots, one to each nostril, a volume of about 0.05 to about 0.25 ml can suitably be administered to each nostril, for a total amount of about 0.1 ml to about 0.5 ml per dose. It is generally desirable to administer as low a volume as practicable, to reduce any tendency for the composition to be partially lost by drainage through the nasopharyngeal passage. Thus particularly suitable volumes are typically about 0.05 to about 0.15 ml per nostril. If desired, however, an entire dose can be administered to one nostril.

As will be clear from the disclosure herein, the composition is useful for administration to subjects of any mammalian species, particularly to human subjects. However, for purposes of defining pharmacokinetic properties of the composition, it will be understood that, except where the context demands otherwise, such properties are stated herein with respect to a rat model, as more fully described in the Example below.

A single dose of the composition, upon intranasal administration in such a rat model, provides a desirable systemic plasma cannabinoid concentration as defined herein. The term “plasma cannabinoid concentration” herein means the total plasma concentration of the tricyclic cannabinoid administered and any tricyclic or non-tricyclic cannabinoid metabolite or metabolites thereof. Intranasal administration of a composition of the invention provides, in the rat model, a plasma cannabinoid concentration of at least about 0.5 ng/ml, for example at least about 1 ng/ml or at least about 5 ng/ml, at least at one time point during a period from about 15 minutes to about 2 hours post-administration, but at no time post-administration does the concentration provided by the composition exceed about 100 ng/ml. Intranasal administration of a single dose of the composition thus does not produce an early, greater than about 100 ng/ml, plasma concentration peak that may be observed with other modes of administration. Instead, the composition is absorbed relatively steadily over time, effectively resulting in a more constant plasma cannabinoid concentration.

In some embodiments, the plasma cannabinoid concentration attains at least about 0.5 ng/ml within about 1 hour, for example within about 30 minutes, post-administration. In other embodiments, the plasma cannabinoid concentration remains no lower than about 0.5 ng/ml for a post-administration period that is variously from about 1 hour to about 2 hours, from about 30 minutes to about 2 hours, from about 30 minutes to about 4 hours, from about 30 minutes to about 6 hours, or from about 30 minutes to about 8 hours.

In further embodiments, the plasma cannabinoid concentration attains at least about 1 ng/ml within about 1 hour, for example within about 30 minutes, post-administration. In still further embodiments, the plasma cannabinoid concentration remains no lower than about 1 ng/ml for a post-administration period that is variously from about 1 hour to about 2 hours, from about 30 minutes to about 2 hours, from about 30 minutes to about 4 hours, from about 30 minutes to about 6 hours, or from about 30 minutes to about 8 hours.

In still further embodiments, the plasma cannabinoid concentration attains at least about 5 ng/ml within about 1 hour, for example within about 30 minutes, post-administration. In still further embodiments, the plasma cannabinoid concentration remains no lower than about 5 ng/ml for a post-administration period that is variously from about 1 hour to about 2 hours, from about 30 minutes to about 2 hours, from about 30 minutes to about 4 hours, from about 30 minutes to about 6 hours, or from about 30 minutes to about 8 hours.

In a still further embodiment, a plasma cannabinoid concentration of at least about 10 ng/ml, for example at least about 30 ng/ml is attained within about 2 hours post-administration.

Bioavailability is a measure of the amount of drug reaching systemic circulation when administered by a route of interest, relative to the amount of drug reaching systemic circulation when administered intravenously. Bioavailability can be represented by calculating the value of the parameter F, which compares the area under the curve (AUC) of plasma concentration after intravenous and intranasal administration in different doses, adjusted for the dose difference. A more detailed understanding of F will be obtainable from the Example below.

It is generally desirable that onset of therapeutic benefit of intranasal administration of a tricyclic cannabinoid such as Δ⁹-THC should occur as soon as possible after administration. Thus in some embodiments absorption of the cannabinoid in the human or non-human subject occurs sufficiently rapidly to enable onset of therapeutic benefit, for example relief of pain or nausea, within about 1 hour, within about 30 minutes or within about 15 minutes.

Independently of onset time, it is generally desirable that a therapeutically beneficial effect should be of sufficient duration that administration to the subject can occur with a dosing frequency no greater than about 4 times a day. Thus in some embodiments plasma cannabinoid concentration in the human or non-human subject remains above a therapeutic threshold, for example above about 0.5 ng/ml, above about 1 ng/ml or above about 5 ng/ml, longer than about 2 hours, for example longer than about 3 hours, longer than about 4 hours or longer than about 6 hours, after administration. It will be understood that what constitutes a therapeutic threshold plasma cannabinoid concentration depends on the subject, the particular tricyclic cannabinoid administered and the nature and severity of the condition to be treated, among other factors, but is typically in the range of about 0.5 to about 5 ng/ml.

According to some embodiments, the bioavailability of the composition when administered intranasally varies less from subject to subject when compared to a standard orally administered dosage form, such as, for example, in the case of Δ⁹-THC, Marinol® capsules. The bioavailability of Δ⁹-THC when intranasally administered in a composition of some embodiments of the invention is at least comparable to the bioavailability of Δ⁹-THC attained by smoking marihuana. Advantageously, in some embodiments the bioavailability is greater and/or less variable than that obtained from smoked marihuana. In various embodiments, the cannabinoid exhibits a bioavailability in a rat model of at least about 0.1 when the composition is administered intranasally, for example a bioavailability of at least about 0.2, or a bioavailability of at least about 0.3, as measured by F.

The composition optionally further comprises a receptivity agent. The term “receptivity agent” herein means an agent that, when included in a pharmaceutical composition administered to a subject, is capable of mitigating an undesirable response to the composition at or in proximity to the locus of administration in or on the subject. Specifically when the locus of administration is intranasal, such undesirable responses that can be mitigated can include an involuntary or reflex response such as sneezing, excessive nasal drip or irritation of nasal tissues, and/or a cognitive response, such as to unpleasant taste or odor. A cognitive response can include a conscious or subconscious decision to reduce or end use of the composition, and can thus affect patient compliance. A receptivity agent can mitigate one or more such undesirable responses.

In some embodiments, the receptivity agent comprises an organoleptic enhancing agent. Illustrative examples of organoleptic enhancing agents include natural and/or synthetic sweeteners, flavorants, aromatics, taste-masking compounds, or combinations thereof.

In some embodiments, an organoleptic enhancing agent included as a receptivity agent comprises a sweetener. Illustrative sweeteners include saccharin, aspartame, neotame, cyclamates, glucose, fructose, sucrose, xylitol, tagatose, sucralose, maltitol, isomaltulose, hydrogenated isomaltulose, lactitol, sorbitol, mannitol, trehalose, maltodextrin, polydextrose, glycerin, erythritol, maltol, acesulfame, acesulfame potassium, alitame, neohesperidin dihydrochalcone, stevioside, thaumatin, sugars, or combinations thereof.

In one embodiment, the receptivity agent comprises an agent that can inhibit sneezing, i.e., an antisternutatory agent.

The composition optionally further comprises one or more pharmaceutically acceptable ingredients, for example, ingredients useful as carriers, preservatives, diluents, stabilizers, pH modulating agents, etc. According to one embodiment, the composition comprises at least one preservative. Preservatives can have antimicrobial activity and/or can serve as antioxidants. Illustrative preservatives include but are not limited to butylated hydroxytoluene, butylated hydroxyanisole, or combinations thereof.

Where the composition is formulated in an aqueous medium, it can comprise one or more tonicity modulating agents, for example in an amount that renders the composition substantially isotonic. For example, a saline solution can form the basis of such a composition.

An apparatus of the invention comprises (a) a reservoir containing a composition as described above comprising at least one cannabinoid, (b) an atomization device configured for insertion in a nostril, and (c) means for actuating the device to deliver droplets of the composition to the nostril. Any sprayable liquid composition as described above is useful in the apparatus. The reservoir can, if desired, be provided separately from the atomization device and actuating means, in which case it is typically adapted for coupling to the atomization device and actuating means prior to use, for example immediately prior to use.

As mentioned above, cannabinoids such as Δ⁹-THC are susceptible to photodegradation. It is therefore generally preferred to protect the composition from light during storage and transportation from time of manufacture until time of use. The reservoir of the present apparatus, for example, can be substantially non-translucent, or provided in an outer substantially non-translucent package. Thus in one embodiment the reservoir or outer packaging thereof protects the cannabinoid from photodegradation.

The atomization device can be any device capable of generating droplets of the liquid composition when the composition is supplied from the reservoir, so long as the device can be inserted in a nostril. In one embodiment, the atomization device comprises a nozzle or constricted passage that, when the liquid composition passes through it under pressure, breaks the liquid up into droplets. Any means known in the art for actuating the atomization device can be employed, for example application of pressure as by squeezing the reservoir or depressing a plunger, or in the case of an electrically operated device, activating a switch.

The range of droplet size produced by the apparatus is dependent upon the physical properties of the composition, for example its viscosity, the nature of the atomization device (e.g., size of a nozzle aperture) and the manner in which the device is actuated to discharge the composition. Droplets should generally not be so fine as to form an inhalable aerosol, but not so coarse as to fail to adhere readily to the nasal mucosa.

Optionally, the apparatus is operable to deliver a metered amount of the composition, for example an amount of about 0.05 to about 0.25 ml, more typically about 0.05 to about 0.15 ml, to a nostril. The apparatus is optionally adjustable to deliver different metered amounts. In some embodiments, the apparatus comprises a nasal spray device, or a modification thereof, that is commercially available, such as those sold by Pfeiffer of America, Inc. (Princeton, N.J.) or by Valois of America, Inc. (Greenwich, Conn.).

A method for delivering a tricyclic cannabinoid to a subject comprises intranasally administering a composition of any of the embodiments described above. The subject can be human or non-human; if non-human, the subject can be an animal, e.g., a mammal, of any species, including domestic animals, farm animals, exotic and zoo animals, laboratory animals, etc.

A method for treatment or prevention of a cannabinoid receptor mediated condition or disorder comprises intranasally administering to a subject, for example a subject in need of such treatment or prevention, a therapeutically effective amount of a composition of any of the embodiments described above. The composition can illustratively be administered in an amount providing a dose of the at least one tricyclic cannabinoid, for example Δ⁹-THC, of about 0.01 to about 10 mg/kg body weight of the subject, for example about 0.05 to about 1 mg/kg body weight of the subject. Such a dose can be administered once or repeatedly at a desired frequency, for example about 1 to about 4 times per day. The composition can illustratively be administered to an adult human in an amount providing a dose of about 0.5 to about 50 mg per day, for example about 2 to about 20 mg per day. What constitutes an appropriate daily dose for a human subject depends on a variety of factors, including the particular cannabinoid in the composition and its bioavailability, the age, sex, and body weight of the subject, the condition being treated, and the severity of that condition.

In the case of a psychotropic cannabinoid such as Δ⁹-THC, a dosage amount and frequency that minimizes psychotropic effects while providing therapeutic benefit can be selected by one of skill in the art without undue experimentation. It is believed that a composition adapted for intranasal administration as provided herein can facilitate such selection, by comparison with intravenous administration or smoking that produces an initial spike in plasma cannabinoid concentration, or with oral administration that exposes the cannabinoid to first-pass metabolism.

The cannabinoid receptor mediated condition or disorder.can be one mediated by CB1, CB2 or both, or, even if not directly cannabinoid receptor mediated, can be associated with a cannabinoid receptor mediated condition or disorder. Such conditions and disorders include, without limitation:
ophthalmic conditions, for example, uveoretinitis, uveitis, iritis, cyclitis, choroiditis, chorioretinitis, vitritis, keratitis, conjunctivitis, diabetic retinopathy, glaucoma and macular degeneration;
inflammatory conditions not included above, for example, inflammatory bowel disease, ulcerative colitis, transplant rejection, vasculitis, dermatomyositis, polymyositis, rheumatoid arthritis, ankylosing spondylitis, spondyloarthritis, arthritis associated with gout, osteoarthritis, atherosclerosis, Crohn's disease, Reiter's syndrome, systemic lupus erythematosus, Sjogren's syndrome, Behcet's disease, thyroiditis, psoriasis, eczema, dermatitis, viral encephalitis, allergic rhinitis, allergic conjunctivitis, T-cell mediated hypersensitivity disease, Guillain-Barré syndrome and Wegener's granulomatosis;
degenerative conditions not included above, for example, osteoporosis, multiple sclerosis, spasticity and myasthenia gravis;
conditions and disorders associated with cancer or treatment of cancer, for example, pain, anorexia, emesis and nausea;
conditions and disorders associated with HIV infection and/or AIDS, for example, cancer, infection, pain, anorexia, emesis, and nausea;
conditions and disorders associated with CNS dysfunction, for example, Huntington's chorea, Parkinson's disease, Tourette's syndrome, depression, Alzheimer's disease, dementia, insomnia, schizophrenia and substance abuse;
conditions and disorders associated with pain and/or trauma, for example, migraine, post-surgical pain, traumatic injury and CNS trauma;
pulmonary conditions, for example, asthma, emphysema, chronic pulmonary obstructive disorder, bronchitis and hypoxia;
cardiovascular conditions, for example, ischemia, angina pectoris, dyslipidemia, coronary artery disease, stroke, cerebral apoplexy, hypertension and cardiac arrest;
endocrine disorders, for example, Hashimoto's thyroiditis, hyperthyroidism, hyperglycemia, diabetes mellitus and impaired glucose intolerance;
conditions associated with obesity; and
conditions associated with abnormal electrical discharge from the brain, for example grand mal seizures, migraine and epilepsy.

Cannabinoids can act as agonists or antagonists of cannabinoid receptors in treatment or prevention of any of the above conditions and disorders. However, usefulness of the present compositions is not limited to situations where cannabinoid receptors such as CB1 and/or CB2 can be shown to be involved. At least some cannabinoids can act as N-methyl-d-aspartate (NMDA) receptor antagonists.

Particular classes of human patients having one or more conditions for which the present invention can be particularly helpful include patients with cancer, patients with HIV infection and/or AIDS, patients with autoimmune disorders, obese patients, and patients with cognitive disorders.

### EXAMPLE

The following example is merely illustrative, and not limiting to this disclosure in any way.

A bioavailability study comparing intranasal (IN) with intravenous (IV) administration of Δ⁹-THC was conducted in 11 male Sprague-Dawley rats: 3 received the drug by IV injection in a propylene glycol solution and 8 by IN injection, 3 receiving the drug in a propylene glycol solution and 5 receiving the drug in a 90:10 propylene glycol/ethanol solution.

The rats were weighed, then anesthetized. To prevent nasal drainage into the stomach or mouth, a closed glass tube was surgically inserted into the esophagus to the posterior part of the nasal cavity and ligated, and the nasopalatine passage was closed. The right jugular vein (and left femoral vein of rats receiving IV injection) were exposed, cannulated and ligated, and the jugular and femoral catheters were flushed with 0.9% saline containing 10 units/ml of heparin to maintain patency

The dosing solutions were administered as follows:
IV administration:
Δ⁹-THC concentration: 100-150 mg/ml
Δ⁹-THC dose: 1 mg/kg
Injection volume: 0.0015-0.003 ml
Injection time: 30 second bolus by hand to left femoral vein
Vehicle: propylene glycol
   For example, at 1 mg/kg Δ⁹-THC, a 290 g rat received 0.29 mg Δ⁹-THC. The 0.29 mg was delivered in 0.003 ml of a 145 mg/ml Δ⁹-THC solution.
IN administration:
Δ⁹-THC concentration: 110-150 mg/ml
Δ⁹-THC dose: 10 mg/kg
Injection volume: 8-11 μl per nostril
Injection time: 30 second bolus by hand to each nostril
Vehicle: propylene glycol or 90:10 propylene glycol/ethanol
   For example, at 10 mg/kg Δ⁹-THC, a 290 g rat received 2.9 mg Δ⁹-THC. The 2.9 mg was delivered in two equal 10 μl aliquots (one 10 μl aliquot per nostril) of a 145 mg/ml Δ⁹-THC solution.

The dosing solutions were prepared by measuring out the appropriate amount of Δ⁹-THC into a 3 ml silanized test tube. The Δ⁹-THC was dried and concentrated on a nitrogen evaporator, then reconstituted in 0.05 ml of propylene glycol, or 90:10 propylene glycol/ethanol.

Intranasal administration was accomplished using a 25 μl gas-tight Hamilton syringe with PE-50 tubing that fit into the rat nasal cavity. The rat nasal cavity volume precluded dosing volumes larger than 10-20 μl per nostril.

Intravenous femoral bolus administration was accomplished using a 10 μl gas-tight Hamilton syringe with a sterile 25-gauge needle. After dosing the animal, the femoral line was flushed with 0.2 ml of drug vehicle (propylene glycol) followed by a 0.2 ml flush of 0.9% saline.

Blood samples, each 0.3 ml in volume, were drawn from the jugular vein using an indwelling jugular catheter at specified times before and after drug administration. Each blood sample was transferred to a siliconized 1.5 ml microcentrifuge tube containing heparin to inhibit blood clotting and centrifuged at 12,000 rpm for 3 minutes. The resulting plasma was transferred to a 2 ml silanized autosampler vial using a 200 μl Eppendorf tube and frozen in a mixture of ethanol and dry ice. Samples were stored at −80° C. until analyzed.

Plasma extraction and preparation for HPLC analysis was performed as follows. In a siliconized microcentrifuge tube 50 μl of plasma was added to 250 μl acetonitrile and 250 μl ethyl acetate, vortexed for 30 seconds, then centrifuged for 20 minutes at 12,000 rpm. The supernatant was removed and placed in silanized 3 ml Kimble culture tubes. The organic phase was evaporated to dryness in an evaporator under nitrogen at 37° C., then the sample was reconstituted in 350 μl of acetonitrile and vortexed for 30 seconds. After 10 minutes of sonification, the samples were transferred to HPLC vials containing silanized low volume National Scientific Company inserts with a Teflon-lined screw cap lid. Drug-spiked plasma standards were prepared similarly.

The HPLC conditions were as in Table 1.

TABLE 1
HPLC Conditions

| | |
|---|---|
| HPLC pump and injector | Waters 2690 Separations Module |
| Detector | Waters 996 Photodiode Array Detector |
| Mass spectrometer | Waters ZQ2000 Mass Spectrometer |
| Data collection | Millenium³² Software |
| Column | 2.1 × 150 mm, 5 μm, Waters Symmetry |
| | Analytical C18 column (#WAT056975) |
| Precolumn | 2.1 × 10 mm, 3.5 μm, Symmetry Sentry |
| | (#WAT106127) |
| Mobile phase | Solvent A: acetonitrile with 5% 2 mM |
| | ammonium acetate |
| | Solvent C: 2 mM ammonium acetate with |
| | 5% acetonitrile |
| Flow rate | 0.25 ml/minute |
| Isocratic | 70% acetonitrile: 30% 2 mM ammonium |
| | acetate |
| Wavelength | Data collected from 200 nm to 300 nm |
| Injection volume | 20 μl |
| Column temperature | 35° C. |
| Sample chamber temperature | 12° C. |
| Probe | ESI positive ion mode |
| SIR | THC (315.5), 11-OH-THC (331.5), |
| | 11-carboxy-THC (345.0) |
| Desolvation temperature | 190-200° C. |
| Source temperature | 135-140° C. |
| Desolvation flow | 473 l/hr |
| Cone flow | 30 l/hr |
| Capillary | 4.59 kV |
| Cone | 35.0 V |
| Extractor | 4.76 V |
| RF lens | 0.5 V |
| Run time | 27 minutes |
| Injection vial | 2 ml glass HPLC vial with silanized |
| | low volume insert with Teflon-lined |
| | screw cap |

Samples were assayed after the LC column was equilibrated by pumping mobile phase for at least 60 minutes and the MS was in operate mode for at least 60 minutes. The samples and standards were loaded into the LC/MS as they were prepared.

Data were analyzed from 0 to 480 minutes after administration using Pharsight WinNonlin® Version 3.3 (California). A non-compartmental model was utilized that described the concentration versus time curve by estimating compartmental parameters such as area under the curve (AUC), Cₘₐₓ and Tₘₐₓ. Non-compartmental parameters were estimated using the linear trapezoidal rule. Non-compartment Model No. 200 (extra-vascular input) was used for intranasal data analysis, and Model No. 201 (bolus IV input) was used for intravenous data analysis.

Tables 2 and 3 show the analyzed data.

TABLE 2
Plasma Concentrations of THC

| | Mean plasma concentration of THC (ng/ml) | | |
|---|---|---|---|
| | | Intranasal (10 mg/kg) | |
| | Intravenous (1 mg/kg) | 90:10 Propylene | |
| Time | Propylene glycol | glycol/ethanol | Propylene glycol |
| (minutes) | n = 3 | n = 5 | n = 3 |
| 0 | 0 | 0 | 0 |
| 0.5 | 693 | 0 | 0 |
| 5 | 136 | 0 | 0 |
| 15 | 73 | 24 | 3 |
| 30 | 48 | 43 | 13 |
| 60 | 34 | 59 | 26 |
| 120 | 24 | 75 | 38 |
| 160 | 23 | 54 | 36 |
| 240 | 21 | 24 | 51 |
| 300 | 18 | 20 | 19 |
| 360 | 22 | 19 | 17 |
| 420 | 25 | 16 | 16 |
| 480 | 12 | 11 | 16 |

TABLE 3
Mean Pharmacokinetic Parameters

| | Intravenous | | |
|---|---|---|---|
| | (IV) | Intranasal (IN) | |
| | Propylene | 90:10 Propylene | Propylene |
| Parameter | glycol | glycol/ethanol | glycol |
| No. of Animals | 3 | 5 | 3 |
| Weight (kg) | 0.260 | 0.258 | 0.267 |
| Dose administered (μg) | 258 | 2556 | 2622 |
| Dose (mg/kg) | 0.99 | 9.91 | 9.84 |
| Tₘₐₓ (hours) | 0.0083 | 2.0 | 3.0 |
| Cₘₐₓ (ng/ml) | 693 | 75 | 55 |
| AUC (ng hr/ml) | 314 | 434 | 428 |
| F* | 1 | 0.138 | 0.136 |

*F = (AUC_{IN} × Dose_{IV})/(AUC_{IV} × Dose_{IN})

The data show that intravenous administration of THC results in a spike in plasma concentration within minutes of dosing, drops rapidly, then tapers off over time. Intranasal administration, on the other hand, produces a plasma concentration profile that remains substantially stable over several hours and lacks the early spike seen with intravenous administration.

Bioavailability, as measured by the parameter F, for intranasal by comparison with intravenous administration of Δ⁹-THC was about 0.14 in this study. This is comparable with reported levels of bioavailability of Δ⁹-THC by inhalation of smoked marihuana.

All references cited above are incorporated herein by reference in their entirety.

The words “comprise”, “comprises”, and “comprising” are to be interpreted inclusively rather than exclusively.

What is claimed is:

1. A pharmaceutical composition for intranasal administration to a human or non-human subject, the composition comprising a therapeutically active component that comprises at least one tricyclic cannabinoid in a liquid to semi-solid medium that comprises a pharmaceutically acceptable solubilizing agent in an amount effective to solubilize the cannabinoid, wherein an amount of the composition intranasally administrable as a single dose, upon intranasal administration in a rat model, provides a systemic plasma cannabinoid concentration (i) that, at least at one time point during a period from about 15 minutes to about 2 hours after said administration, is at least about 0.5 ng/ml, but (ii) that at no time exceeds about 100 ng/ml.

2. The composition of claim 1, wherein said plasma cannabinoid concentration attains at least about 0.5 ng/ml within about 30 minutes after said administration.

3. The composition of claim 1, wherein said plasma cannabinoid concentration remains at least about 0.5 ng/ml for a period from about 30 minutes to about 2 hours after said administration.

4. The composition of claim 1, wherein said plasma cannabinoid concentration remains at least about 0.5 ng/ml for a period from about 30 minutes to about 6 hours after said administration.

5. The composition of claim 1, having a bioavailability of at least about 0.1 when intranasally administered in a rat model.

6. The composition of claim 5, wherein said bioavailability is at least about 0.3 when intranasally administered in a rat model.

7. The composition of claim 5, wherein bioavailability exhibits less subject-to-subject variability than a standard orally administered dosage form of the cannabinoid.

8. The composition of claim 1, wherein the at least one tricyclic cannabinoid is a CB1 receptor selective agonist.

9. The composition of claim 1, wherein the at least one tricyclic cannabinoid is a CB2 receptor selective agonist.

10. The composition of claim 1, wherein the at least one tricyclic cannabinoid is hydrophobic.

11. The composition of claim 10, wherein the at least one tricyclic cannabinoid comprises a tetrahydrocannabinol.

12. The composition of claim 10, wherein the at least one tricyclic cannabinoid comprises Δ⁹-THC.

13. The composition of claim 12, wherein the Δ⁹-THC is synthetic.

14. The composition of claim 12, wherein the Δ⁹-THC is present in the composition at a concentration of at least about 1 mg/ml.

15. The composition of claim 12, wherein the Δ⁹-THC is present in the composition at a concentration of about 1 to about 200 mg/ml.

16. The composition of claim 12, wherein the Δ⁹-THC is present in the composition at a concentration of about 5 to about 50 mg/ml.

17. The composition of claim 10, wherein the solubilizing agent comprises at least one glycol.

18. The composition of claim 17, wherein the at least one glycol is selected from the group consisting of propylene glycol, 1,3-butanediol, polyethylene glycol, propylene glycol fatty acid esters, and diethylene glycol monoethyl ether.

19. The composition of claim 17, wherein the at least one glycol is propylene glycol.

20. The composition of claim 19, wherein the solubilizing agent further comprises ethanol.

21. The composition of claim 20, wherein the propylene glycol and ethanol are present in a volume ratio of at least about 80:20.

22. The composition of claim 17, wherein the solubilizing agent is essentially free of ethanol.

23. The composition of claim 10, further comprising water providing an aqueous medium for the cannabinoid, wherein the solubilizing agent comprises at least one amphiphilic compound in an amount effective to solubilize the cannabinoid in the aqueous medium.

24. The composition of claim 23, wherein the at least one amphiphilic compound is a cationic, anionic or nonionic surfactant.

25. The composition of claim 23, wherein the at least one amphiphilic compound is selected from the group consisting of benzalkonium chloride, benzethonium chloride, cetylpyridinium chloride, dioctyl sodium sulfosuccinate, nonoxynol 9, nonoxynol 10, octoxynol 9, poloxamers, polyoxyethylene (8) caprylic/capric mono- and diglycerides, polyoxyethylene (35) castor oil, polyoxyethylene (20) cetostearyl ether, polyoxyethylene (40) hydrogenated castor oil, polyoxyethylene (10) oleyl ether, polyoxyethylene (40) stearate, polysorbate 20, polysorbate 40, polysorbate 60, polysorbate 80, propylene glycol laurate, sodium lauryl sulfate, sorbitan monolaurate, sorbitan monooleate, sorbitan monopalmitate, sorbitan monostearate and tyloxapol, and mixtures thereof.

26. The composition of claim 1, further comprising at least one receptivity agent.

27. The composition of claim 26, wherein the at least one receptivity agent is an organoleptic enhancing agent.

28. The composition of claim 27, wherein the organoleptic enhancing agent is selected from the group consisting of natural sweeteners, synthetic sweeteners, flavorants, aromatics, taste-masking compounds, and combinations thereof.

29. The composition of claim 27, wherein the organoleptic enhancing agent is a sweetener selected from the group consisting of saccharin, aspartame, neotame, cyclamates, glucose, fructose, sucrose, xylitol, tagatose, sucralose, maltitol, isomaltulose, hydrogenated isomaltulose, lactitol, sorbitol, mannitol, trehalose, maltodextrin, polydextrose, glycerin, erythritol, maltol, acesulfame, acesulfame potassium, alitame, neohesperidin dihydrochalcone, stevioside, thaumatin, sugars and combinations thereof.

30. The composition of claim 1, further comprising at least one preservative or antioxidant.

31. The composition of claim 30, wherein the at least one preservative or antioxidant is selected from the group consisting of butylated hydroxytoluene, butylated hydroxyanisole and combinations thereof.

32. The composition of claim 1, wherein the composition comprises a sufficiently low level of oxidatively reactive substances and/or a sufficiently high level of one or more antioxidants to result in zero to an acceptably low degree of oxidative degradation of the cannabinoid under normal storage conditions in a sealed lightproof container.

33. The composition of claim 1, wherein the medium is semi-solid and the composition is a cream, gel, or ointment.

34. The composition of claim 1, wherein said medium is liquid and the composition is sprayable.

35. An apparatus comprising (a) a reservoir containing a composition of claim 34, (b) an atomization device configured for insertion in a nostril, and (c) means for actuating said device to deliver droplets of said composition to the nostril.

36. The apparatus of claim 35, that is operable to deliver a metered amount of said composition to the nostril.

37. The apparatus of claim 36, wherein said metered amount is about 0.05 to about 0.25 ml.

38. The apparatus of claim 35 wherein the reservoir or outer packaging thereof protects the cannabinoid from photodegradation.

39. A method for delivering a cannabinoid to a subject, the method comprising intranasally administering a composition of claim 1.

40. The method of claim 39, wherein the subject is a patient with cancer, with HIV infection and/or AIDS, with an autoimmune disorder or with a cognitive disorder, or an obese patient.

41. A method for treatment or prevention of a cannabinoid receptor mediated condition or disorder, the method comprising intranasally administering to a subject a therapeutically effective amount of a composition of claim 1.

42. The method of claim 41, wherein the subject is a human patient.

43. The method of claim 42, wherein the composition is administered in an amount providing a dose of the at least one tricyclic cannabinoid of about 0.5 to about 50 mg per day.

44. The method of claim 43, wherein the composition is administered in an amount providing a dose of the at least one tricyclic cannabinoid of about 2 to about 20 mg per day.

45. The method of claim 43, wherein the condition or disorder is selected from the group consisting of ophthalmic conditions, inflammatory conditions, degenerative conditions; conditions and disorders associated with cancer or treatment of cancer;
conditions and disorders associated with HIV infection and/or AIDS, conditions and disorders associated with CNS dysfunction, conditions and disorders associated with pain and/or trauma, pulmonary conditions, cardiovascular conditions, endocrine disorders, conditions associated with obesity, and conditions associated with abnormal electrical discharge from the brain.

46. The method of claim 43, wherein the condition or disorder is selected from the group consisting of uveoretinitis, uveitis, iritis, cyclitis, choroiditis, chorioretinitis, vitritis, keratitis, conjunctivitis, diabetic retinopathy, glaucoma, macular degeneration, inflammatory bowel disease, ulcerative colitis, transplant rejection, vasculitis, dermatomyositis, polymyositis, rheumatoid arthritis, ankylosing spondylitis, spondyloarthritis, arthritis associated with gout, osteoarthritis, atherosclerosis, Crohn's disease, Reiter's syndrome, systemic lupus erythematosus, Sjogren's syndrome, Behcet's disease, thyroiditis, psoriasis, eczema, dermatitis, viral encephalitis, allergic rhinitis, allergic conjunctivitis, T-cell mediated hypersensitivity disease, Guillain-Barré syndrome, Wegener's granulomatosis, osteoporosis, multiple sclerosis, spasticity, myasthenia gravis, pain, anorexia, emesis, nausea, Huntington's chorea, Parkinson's disease, Tourette's syndrome, depression, Alzheimer's disease, dementia, insomnia, schizophrenia, substance abuse, migraine, post-surgical pain, traumatic injury, CNS trauma, asthma, emphysema, chronic pulmonary obstructive disorder, bronchitis, hypoxia, ischemia, angina pectoris, dyslipidemia, coronary artery disease, stroke, cerebral apoplexy, hypertension, cardiac arrest, Hashimoto's thyroiditis, hyperthyroidism, hyperglycemia, diabetes mellitus, impaired glucose intolerance, grand mal seizures, migraine and epilepsy.
